(19) United States
(12) Patent Application Publication
(10) Pub. No.: US 2023/0316716 A1
(43) Pub. Date: Oct. 5, 2023

(54) SYSTEMS AND METHODS FOR AUTOMATED LESION DETECTION USING MAGNETIC RESONANCE FINGERPRINTING DATA

(71) Applicants: Case Western Reserve University, Cleveland, OH (US); The Cleveland Clinic Foundation, Cleveland, OH (US)

(72) Inventors: Dan Ma, Solon, OH (US); Zhong Wang, Pepper Pike, OH (US)

(21) Appl. No.: 18/178,120

(22) Filed: Mar. 3, 2023

Related U.S. Application Data
(60) Provisional application No. 63/268,841, filed on Mar. 3, 2022.

(51) Int. Cl. G06V 10/764; G06T 7/00; G06T 7/32

(52) U.S. Cl. CPC G06V 10/764; G06T 7/0012; G06T 7/32; G06T 2207/10088; G06T 2207/20081; G06T 2207/30016; G06T 2207/30096

(57) ABSTRACT

Systems and methods for automated lesion detection are provided. Quantitative maps of tissue properties may be generated from magnetic resonance fingerprinting (MRF) data and may be used with a z-score determination and a trained lesion detection classifier for automated epilepsy lesion detection. In some configurations, lesion detection may be performed at an individual-level with MRF data associated with a subject that includes a detectable lesion.

### CROSS-REFERENCE TO RELATED APPLICATIONS

The present application is based on, claims priority to, and incorporates herein by reference in its entirety U.S. Provisional Pat. Application No. 63/268,841, filed Mar. 3, 2022, and entitled “SYSTEMS AND METHODS FOR AUTOMATED LESION DETECTION USING MAGNETIC RESONANCE FINGERPRINTING DATA.”

### STATEMENT REGARDING FEDERALLY SPONSORED RESEARCH

This invention was made with government support under R01 NS109439 awarded by National Institutes of Health. The government has certain rights in the invention.

### BACKGROUND

The present disclosure relates generally to medical imaging and, more particularly, the present disclosure relates to systems and methods for automated lesion detection using magnetic resonance (MR) data, including MR fingerprinting (MRF) data.

Epilepsy is a neurological disorder where brain activity becomes abnormal, causing seizures or periods of unusual behavior, sensations, and sometimes loss of awareness. Epilepsy affects both males and females of all races, ethnic backgrounds, and ages.

Focal cortical dysplasia (FCD) is one of the most common underlying pathologies for medically intractable epilepsies. Surgical resection/ablation of the FCD lesion is the most effective intervention to achieve seizure-freedom. Although a fair percentage of lesions can be visually appreciated on conventional magnetic resonance imaging (MRI), it is challenging to distinguish subtle lesions from normal brain tissues, partly due to a lack of sensitive and specific MRI measurements.

Previous attempts to correct the shortcomings of conventional MRI have included voxel-based postprocessing based on conventional T1-weighted MRI images. These methods have been performed to detect lesions in individual epilepsy patients, suggesting advanced post-processing of MRI can improve diagnostic yield and accuracy. One example of a post-processing method includes a voxel-based post-processing technique that extracts gray matter (GM) and white matter (WM) maps from individuals to make statistical comparisons with respect to a normal database. Voxel-based post-processing techniques have shown to be effective in detecting epilepsy lesions, but these methods still suffer from the fundamental shortcomings associated with the traditional MRI data they rely upon, and thus still lack sensitivity to subtle lesions.

Thus, there remains a need for systems and methods capable of distinguishing lesions from normal tissue with increased sensitivity and efficacy than is possible with conventional MRI measurements.

### SUMMARY OF THE DISCLOSURE

The present disclosure addresses the aforementioned drawbacks by providing systems and methods for epilepsy lesion detection that can detect lesions that are not generally discerned using traditional MR data and post-processing methods. The systems and methods provided herein may create quantitative maps of tissue properties that may be generated from magnetic resonance fingerprinting (MRF) data. In one non-limiting example, a z-score determination may be created that can facilitate detection of lesions, including epilepsy lesions, that are not generally detected with traditional MR data, including T1-weighted imaging. In some aspects of the present disclosure, a trained lesion detection classifier may be used for automated epilepsy lesion detection. In some configurations, automated epilepsy lesion detection may be performed at an individual-level with MRF data associated with a subject that includes a detectable epilepsy lesion.

In one aspect, a method is provided for automatically detecting a lesion in magnetic resonance fingerprinting (MRF) data of a subject. The method includes accessing MRF data of a subject containing the lesion and registering the MRF data of the subject to a template space. The method also includes generating a normal template from normal images without a lesion in the template space. The method also includes generating a z-score map using the registered MRF data of the subject and the generated normal template. The method also includes subjecting the generated z-score map to a classifier trained to detect the lesion in the generated z-score map and displaying an image of the subject with the detected lesion.

In one aspect, a system is provided for automatically detecting a lesion in magnetic resonance fingerprinting (MRF) data of a subject. The system includes a computer system configured to: i) access MRF data of a subject containing the lesion; ii) register the MRF data of the subject to a template space; iii) generate a normal template from normal images without a lesion in the template space; iv) generate a z-score map using the registered MRF data of the subject and the generated normal template; v) subject the generated z-score map to a classifier trained to detect the lesion in the generated z-score map; and vi) display an image of the subject with the detected lesion.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of an example magnetic resonance imaging (“MRI”) system that can implement the methods described in the present disclosure.

FIG. 2 is a flowchart of non-limiting example steps for an automated method of epilepsy lesion detection in accordance with the present disclosure.

FIG. 3 is another flowchart of non-limiting example steps for automated epilepsy lesion detection.

FIG. 4 is a flowchart of non-limiting example steps for an automated lesion detection based upon generated z-score maps.

FIG. 5 depicts non-limiting example Magnetic Resonance Fingerprinting (MRF) Data, including T1, T2, GM and WM maps.

FIG. 6 depicts non-limiting example original and processed images from an epilepsy patient with automatically identified lesions in accordance with the present disclosure.

FIG. 7 is a block diagram illustrating a system in accordance with the present disclosure.

FIG. 8 is another block diagram illustrating a system in accordance with the present disclosure.

### DETAILED DESCRIPTION

Systems and methods for automated epilepsy lesion detection are provided. Quantitative maps of tissue properties may be generated from MRF data and may be used with a z-score determination and a trained lesion detection classifier for automated epilepsy lesion detection. In some configurations, lesion detection may be performed at an individual-level with MRF data associated with a subject that includes a detectable epilepsy lesion.

Magnetic Resonance Fingerprinting (MRF) is an MRI technique that makes it possible to measure whole-brain quantitative tissue property values, e.g., T1 map, T2 map, M0 (proton density) map, GM map, WM map, and the like. Since FCD causes abnormalities in both cyto- and myelo-architectures of the cortex, direct measurement of tissue properties is highly relevant for detecting FCD lesions, especially subtle ones that would not be noticeable with conventional MRI.

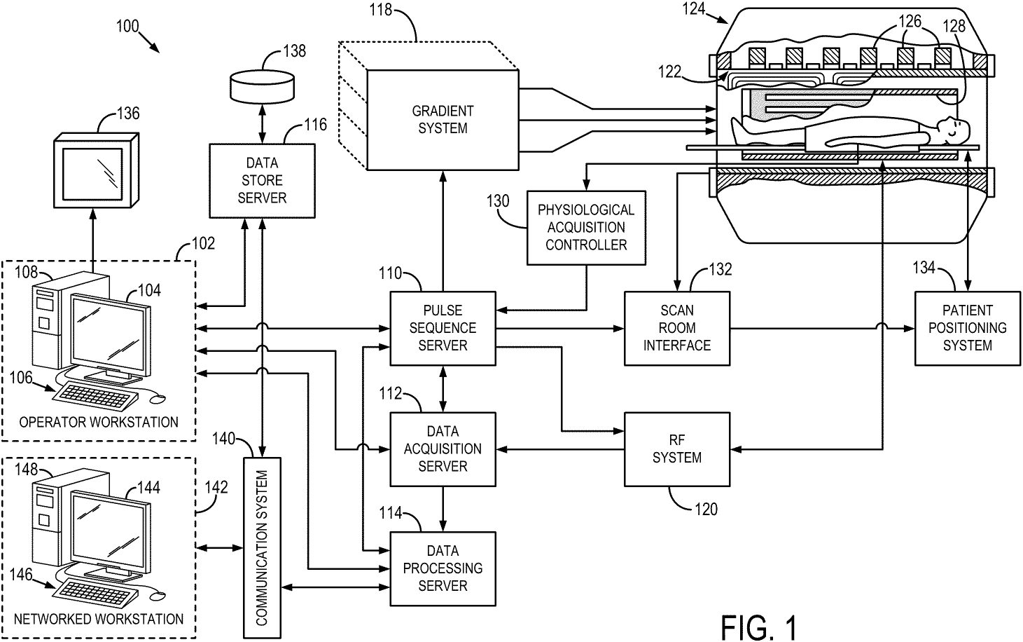

Referring particularly now to FIG. 1, an example of an MRI system 100 that can implement the methods described here is illustrated. The MRI system 100 includes an operator workstation 102 that may include a display 104, one or more input devices 106 (e.g., a keyboard, a mouse), and a processor 108. The processor 108 may include a commercially available programmable machine running a commercially available operating system. The operator workstation 102 provides an operator interface that facilitates entering scan parameters into the MRI system 100. The operator workstation 102 may be coupled to different servers, including, for example, a pulse sequence server 110, a data acquisition server 112, a data processing server 114, and a data store server 116. The operator workstation 102 and the servers 110, 112, 114, and 116 may be connected via a communication system 140, which may include wired or wireless network connections.

The pulse sequence server 110 functions in response to instructions provided by the operator workstation 102 to operate a gradient system 118 and a radiofrequency (“RF”) system 120. Gradient waveforms for performing a prescribed scan are produced and applied to the gradient system 118, which then excites gradient coils in an assembly 122 to produce the magnetic field gradients Gₓ, G_{y} and G_{z} that are used for spatially encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil to perform the prescribed magnetic resonance pulse sequence. Responsive magnetic resonance signals detected by the RF coil 128, or a separate local coil, are received by the RF system 120. The responsive magnetic resonance signals may be amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the prescribed scan and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. An RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} \qquad \text{(1)}$$

and the phase of the received magnetic resonance signal may also be determined according to the following relationship:

$$φ = {tan}^{- 1} \left(\frac{Q}{I}\right) \qquad \text{(2)}$$

The pulse sequence server 110 may receive patient data from a physiological acquisition controller 130. By way of example, the physiological acquisition controller 130 may receive signals from a number of different sensors connected to the patient, including electrocardiograph (“ECG”) signals from electrodes, or respiratory signals from a respiratory bellows or other respiratory monitoring devices. These signals may be used by the pulse sequence server 110 to synchronize, or “gate,” the performance of the scan with the subject’s heart beat or respiration.

The pulse sequence server 110 may also connect to a scan room interface circuit 132 that receives signals from various sensors associated with the condition of the patient and the magnet system. Through the scan room interface circuit 132, a patient positioning system 134 can receive commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, so that data is not lost by data overrun. In some scans, the data acquisition server 112 passes the acquired magnetic resonance data to the data processor server 114. In scans that require information derived from acquired magnetic resonance data to control the further performance of the scan, the data acquisition server 112 may be programmed to produce such information and convey it to the pulse sequence server 110. For example, during pre-scans, magnetic resonance data may be acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also process magnetic resonance signals used to detect the arrival of a contrast agent in a magnetic resonance angiography (“MRA”) scan. For example, the data acquisition server 112 may acquire magnetic resonance data and processes it in real-time to produce information that is used to control the scan.

The data processing server 114 receives magnetic resonance data from the data acquisition server 112 and processes the magnetic resonance data in accordance with instructions provided by the operator workstation 102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 114 are conveyed back to the operator workstation 102 for storage. Real-time images may be stored in a data base memory cache, from which they may be output to operator display 102 or a display 136. Batch mode images or selected real time images may be stored in a host database on disc storage 138. When such images have been reconstructed and transferred to storage, the data processing server 114 may notify the data store server 116 on the operator workstation 102. The operator workstation 102 may be used by an operator to archive the images, produce films, or send the images via a network to other facilities.

The MRI system 100 may also include one or more networked workstations 142. For example, a networked workstation 142 may include a display 144, one or more input devices 146 (e.g., a keyboard, a mouse), and a processor 148. The networked workstation 142 may be located within the same facility as the operator workstation 102, or in a different facility, such as a different healthcare institution or clinic.

The networked workstation 142 may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be remotely processed by a networked workstation 142.

The above-described system can be used to perform a magnetic resonance fingerprinting (“MRF”) process in accordance with the present disclosure. MRF is a technique that facilitates determining and mapping of tissue or other material properties based on measurements of the subject or object being imaged. In particular, MRF can be conceptualized as employing a series of varied “sequence blocks” that simultaneously produce different signal evolutions in different “resonant species” to which the RF is applied. The term “resonant species,” as used herein, refers to a material, such as water, fat, bone, muscle, soft tissue, and the like, that can be made to resonate using NMR. By way of illustration, when radio frequency (“RF”) energy is applied to a volume that has both bone and muscle tissue, then both the bone and muscle tissue will produce a nuclear magnetic resonance (“NMR”) signal; however, the “bone signal” represents a first resonant species and the “muscle signal” represents a second resonant species, and thus the two signals will be different. These different signals from different species can be collected simultaneously over a period of time to collect an overall “signal evolution” for the volume.

The measurements obtained in MRF techniques are achieved by varying the acquisition parameters from one repetition time (“TR”) period to the next, which creates a time series of signals with varying contrast. Examples of acquisition parameters that can be varied include flip angle (“FA”), RF pulse phase, TR, echo time (“TE”), and sampling patterns, such as by modifying one or more readout encoding gradients. In some cases the varied acquisition parameters may be varied in a random manner, pseudorandom manner, or other pattern or manner that results in signals from different materials or tissues to be spatially incoherent, temporally incoherent, or both. For example, in some instances, the acquisition parameters can be varied according to a non-random or non-pseudorandom pattern that otherwise results in signals from different materials or tissues to be spatially incoherent, temporally incoherent, or both.

From these measurements, which may contain signals from different materials or tissues that are spatially incoherent, temporally incoherent, or both, MRF processes can be designed to map any of a wide variety of parameters. Examples of such parameters that can be mapped may include, but are not limited to, longitudinal relaxation time (T₁), transverse relaxation time (T₂ ), main or static magnetic field map ( B₀), and proton density (ρ). MRF is generally described in U.S. Pat. No. 8,723,518 and U.S. Pat. No. 10,261,154, each of which is incorporated herein by reference in its entirety.

The data acquired with MRF techniques are compared with a dictionary of signal models, or templates, that have been generated for different acquisition parameters from magnetic resonance signal models, such as Bloch equation-based physics simulations. This comparison allows estimation of the physical parameters, such as those mentioned above. As an example, the comparison of the acquired signals to a dictionary can be performed using any suitable matching or pattern recognition technique. The parameters for the tissue or other material in a given voxel are estimated to be the values that provide the best signal template matching. For instance, the comparison of the acquired data with the dictionary can result in the selection of a signal vector, which may constitute a weighted combination of signal vectors, from the dictionary that best corresponds to the observed signal evolution. The selected signal vector includes values for multiple different quantitative parameters, which can be extracted from the selected signal vector and used to generate the relevant quantitative parameter maps.

The stored signals and information derived from reference signal evolutions may be associated with a potentially very large data space. The data space for signal evolutions can be partially described by:

$$S E = {\sum }_{s = 1}^{{N}_{S}} {\prod }_{i = 1}^{{N}_{A}} {\sum }_{j = 1}^{{N}_{R F}} {R}_{i} \left(α\right) {R}_{R {F}_{i j}} \left(α , ϕ\right) R \left(G\right) {E}_{i} \left({T}_{1} , {T}_{2} , D\right) {M}_{0} \qquad \text{(3)}$$

where SE is a signal evolution; Nₛ is a number of spins; N_{A} is a number of sequence blocks; N_{RF} is a number of RF pulses in a sequence block; α is a flip angle; ø is a phase angle; R_{¡}(α) is a rotation due to off resonance; R_{RFᵢⱼ} (α,ø) is a rotation due to RF differences; R(G) is a rotation due to a magnetic field gradient; T₁ is a longitudinal, or spin-lattice, relaxation time; T₂ is a transverse, or spin-spin, relaxation time; D is diffusion relaxation; Eᵢ (T₁,T₂,D) is a signal decay due to relaxation differences; and M ₀ is the magnetization in the default or natural alignment to which spins align when placed in the main magnetic field.

While Eᵢ (T₁,T₂,D) is provided as an example, in different situations, the decay term, Eᵢ (T₁,T₂,D), may also include additional terms, Eᵢ (T₁, T₂, D,...) or may include fewer terms, such as by not including the diffusion relaxation, as Eᵢ (T₁,T₂) or Eᵢ (T₁,T₂,...) Also, the summation on “j” could be replace by a product on “j”.

The dictionary may store signals described by,

$${S}_{i} = {R}_{i} {E}_{i} \left({S}_{i - 1}\right) \qquad \text{(4)}$$

where S₀ is the default, or equilibrium, magnetization; Sᵢ is a vector that represents the different components of magnetization, M ₓ, M_{y}, and M _{z} during the i^{th} acquisition block; Rᵢ is a combination of rotational effects that occur during the i^{th} acquisition block; and Eᵢ is a combination of effects that alter the amount of magnetization in the different states for the i^{th} acquisition block. In this situation, the signal at the i^{th} acquisition block is a function of the previous signal at acquisition block (i.e., the (i - 1)^{th} acquisition block). Additionally or alternatively, the dictionary may store signals as a function of the current relaxation and rotation effects and of previous acquisitions. Additionally or alternatively, the dictionary may store signals such that voxels have multiple resonant species or spins, and the effects may be different for every spin within a voxel. Further still, the dictionary may store signals such that voxels may have multiple resonant species or spins, and the effects may be different for spins within a voxel, and thus the signal may be a function of the effects and the previous acquisition blocks.

As described above, data acquired with an MRF technique generally includes data containing random measurements, pseudorandom measurements, or measurements obtained in a manner that results in spatially incoherent signals, temporal incoherent signals, or spatiotemporally incoherent signals. For instance, such data can be acquired by varying acquisition parameters from one TR period to the next, which creates a time series of signals with varying contrast. Using this series of varied sequence blocks simultaneously produces different signal evolutions in different resonant species to which RF energy is applied.

As an example, data are acquired using a pulse sequence where effectuating the pulse sequence includes controlling an NMR apparatus (e.g., an MRI system) to apply RF energy to a volume in an object being imaged. The volume may contain one or more resonant species, such as tissue, fat, water, hydrogen, and prosthetics.

The RF energy may be applied in a series of variable sequence blocks. Sequence blocks may vary in a number of parameters including, but not limited to, echo time, flip angle, phase encoding, diffusion encoding, flow encoding, RF pulse amplitude, RF pulse phase, number of RF pulses, type of gradient applied between an excitation portion of a sequence block and a readout portion of a sequence block, number of gradients applied between an excitation portion of a sequence block and a readout portion of a sequence block, type of gradient applied between a readout portion of a sequence block and an excitation portion of a sequence block, number of gradients applied between a readout portion of a sequence block and an excitation portion of a sequence block, type of gradient applied during a readout portion of a sequence block, number of gradients applied during a readout portion of a sequence block, amount of RF spoiling, and amount of gradient spoiling. Depending upon the imaging or clinical need, two, three, four, or more parameters may vary between sequence blocks. The number of parameters varied between sequence blocks may itself vary. For example, a first sequence block may differ from a second sequence block in five parameters, the second sequence block may differ from a third sequence block in seven parameters, the third sequence block may differ from a fourth sequence block in two parameters, and so on. One skilled in the art will appreciate that there are a very-large number of series of sequence blocks that can be created by varying this large number of parameters. A series of sequence blocks can be crafted so that the series have different amounts (e.g., 1%, 2%, 5%, 10%, 50%, 99%, 100%) of unique sequence blocks as defined by their varied parameters. A series of sequence blocks may include more than ten, more than one hundred, more than one thousand, more than ten thousand, and more than one hundred thousand sequence blocks. In one example, the only difference between consecutive sequence blocks may be the number or parameters of excitation pulses.

Regardless of the particular imaging parameters that are varied or the number or type of sequence blocks, the RF energy applied during a sequence block is configured to cause different individual resonant species to simultaneously produce individual NMR signals. Unlike conventional imaging techniques, in an MRF pulse sequence, at least one member of the series of variable sequence blocks will differ from at least one other member of the series of variable sequence blocks in at least N sequence block parameters, where N is an integer greater than one. One skilled in the art will appreciate that the signal content of a signal evolution may vary directly with N. Thus, as more parameters are varied, a potentially richer signal is retrieved. Conventionally, a signal that depends on a single parameter is desired and required to facilitate imaging. Here, acquiring signals with greater information content facilitates producing more distinct, and thus more matchable, signal evolutions.

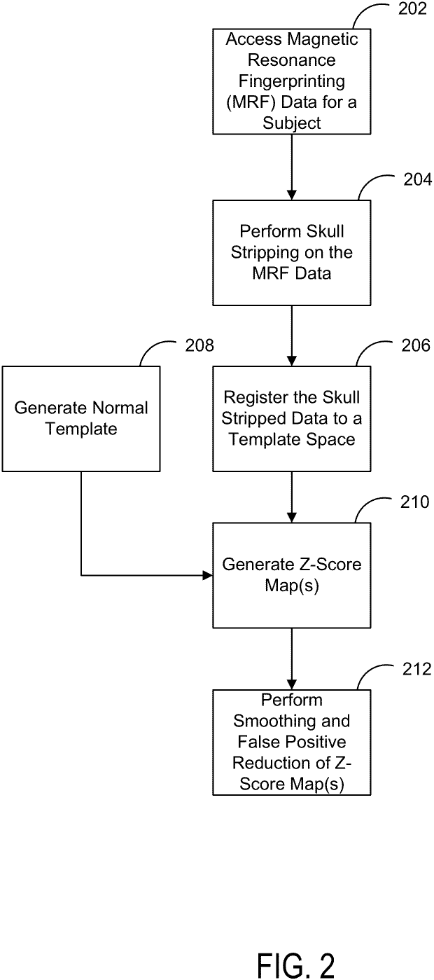

Referring to FIG. 2, non-limiting example steps for automated epilepsy lesion detection are shown. MRF data of a subject may be accessed or acquired at step 202. MRF data may be acquired using an MRI system with sequence blocks as described above. MRF data may be accessed from an image storage archive, such as a picture archiving and communication system (PACS), which may include accessing stored MRF or MRI images of a subject.

The MRF data may include a skull of a subject, and the skull may be stripped from the MRF data at step 204. Skull stripping may be performed on any MRF images, such as T1, T2, gray matter (GM), white matter (WM), other MRF maps, and the like, from patients or normal controls. Previously, conventional MRI images were used as input images, which are not quantitative images, and therefore these previous methods would not work with MRF images. In accordance with the present disclosure, a weighted sum of both an MRF T1 map and MRF synthesized T1w images may be used to best segment the skull tissues. Once the skull tissues are extracted, a “brain mask” may be created and applied on all MRF images so that only tissue within the brain is considered for the following steps.

Registration of the skull-stripped data to a template space may be performed at step 206. The skull-stripped data or images may be registered to a template space to provide for comparison with normal controls. MRF synthesized T1w images may be used together with MRF maps to provide for optimized registration results.

A normal template may be generated at step 208. Normalized control images may be used to generate a template image that has mean and standard deviation maps over a normal range. The process may be repeated by performing registration between images to generate a fine-tuned template image, rather than simply calculating a mean or standard deviation across data.

A z-score map may be generated at step 210. Generating z-score maps, such as a whole-brain, voxel-wise z-score map for an individual patient may be generated using the following expression:

$${Z}_{s c o r e} = \frac{M R {F}_{N} - M R {F}_{P}}{M R {F}_{S D}} \qquad \text{(5)}$$

Where Z_{score} represents a pixel value in a z-score map, MRF_{N} is the mean of a normal MRF value determined from the normal template, MRF_{P} is a subject or patient MRF value, and MRF_{SD} is the standard deviation of the normal template MRF values. Previous z-score calculations have been based on morphologic feature maps of the brain, such as cortical thickness or shape. In accordance with the present disclosure, MRF values, such as T1, T2, M0, GM, WM and the like, may be used in determining z-score values. In some configurations, multiple, quantitative MRF maps may be used to generate the z-score maps for lesion detection. Expressions for z-score determinations from non-limiting example quantitative tissue properties include:

$${Z}_{s c o r e} = \frac{{T}_{1 P} - {T}_{1 N}}{{T}_{1 N - S D}} \qquad \text{(6)}$$

Where T_{1N} is the mean of a normal T₁ MRF value determined from the normal template, T_{1P} is a subject or patient T₁ MRF value, and T_{1N}-_{SD} is the standard deviation of the normal template T₁ MRF values.

$${Z}_{s c o r e} = \frac{{T}_{2 P} - {T}_{2 N}}{{T}_{2 N - S D}} \qquad \text{(7)}$$

Where T_{2N} is the mean of a normal T₂ MRF value determined from the normal template, T_{2P} is a subject or patient T₂ MRF value, and T_{2N-SD} is the standard deviation of the normal template T₂ MRF values.

$${Z}_{s c o r e} = \frac{G {M}_{P} - G {M}_{N}}{G {M}_{N - S D}} \qquad \text{(8)}$$

Where GM_{N} is the mean of a normal GM MRF value determined from the normal template, GM_{P} is a subject or patient GM MRF value, and GM_{N-SD} is the standard deviation of the normal template GM MRF values.

$${Z}_{s c o r e} = \frac{W {M}_{P} - W {M}_{N}}{W {M}_{N - S D}} \qquad \text{(9)}$$

Where WM_{N} is the mean of a normal WM MRF value determined from the normal template, WM_{P} is a subject or patient WM MRF value, and WM_{N-SD} is the standard deviation of the normal template WM MRF values.

Referring still to FIG. 2, smoothing and false positive reduction may be performed at step 212. In a non-limiting example, Gaussian smoothing may be applied to the z-score maps to reduce the noise level. Cerebrospinal fluid (CSF) masks, which may be generated by MRF CSF maps and tissue segmentation of the MRF synthesized T₁ weighted images, may be used to exclude CSF components at the tissue border, thereby reducing false-positives.

In some configurations, the z-score maps can be used independently of each other to support visual inspection of the images by a radiologist or neurologist, as the maps contain complimentary tissue property information. In a non-limiting example, one map can be useful for one type of pathology, while another map can be useful for a different pathology.

In some configurations, the methods in accordance with the present disclosure can be practically used in the clinical setting for presurgical evaluation of individual epilepsy patients. Detection of lesions, or delineating extent of lesions may also be provided. These aspects are key to success of surgical planning of epilepsy patients.

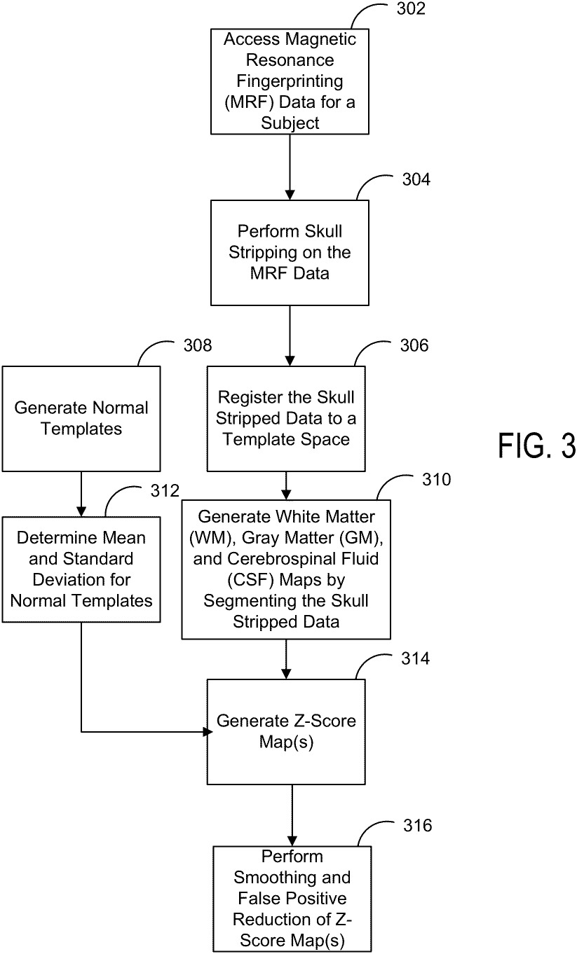

Referring to FIG. 3, another non-limiting example steps for automated epilepsy lesion detection are shown. MRF data of a subject may be accessed or acquired at step 302. As described above, MRF data may be acquired using an MRI system with sequence blocks or may be accessed from an image storage archive. The MRF data may include a skull of a subject, and the skull may be stripped from the MRF data at step 304, as described above. Registration of the skull-stripped data to a template space may be performed at step 306. The skull-stripped data or images may be registered to a template space to provide for comparison with normal controls.

Normal templates may be generated at step 308. Normalized control images may be used to generate template images that have mean and standard deviation maps to establish a normal range. At step 312, the mean and standard deviation of the normal templates are determined. The process may be repeated by performing registration between images to generate a fine-tuned template image, rather than simply calculating a mean or standard deviation across data.

WM, GM, and CSF maps may be generated by segmenting the skull-stripped data at step 310. Segmenting may be performed by identifying the tissues in the skull-stripped data such as by identifying the MRF values for each pixel and segmenting pixels of similar values, within a specified range. A z-score map may then be generated at step 314 by considering the mean and standard deviation maps generated from the normal templates and the WM, GM, and CSF maps using the expressions disclosed above. Z-score maps, such as a whole-brain, voxel-wise z-score maps for an individual patient may be generated.

Smoothing and false positive reduction may be performed at step 316 for the generated z-score maps. In a non-limiting example, Gaussian smoothing may be applied to the z-score maps to reduce the noise level. Cerebrospinal fluid (CSF) masks, which may be generated by MRF CSF maps and tissue segmentation of the MRF synthesized T₁ weighted images, may be used to exclude CSF components at the tissue border, thereby reducing false-positives.

In some configurations, information from all maps can be combined. Training of a machine learning or deep learning classifier, or the like can be performed based on labeled patient data and control data. The classifier can produce a final probability map indicating the detected lesion.

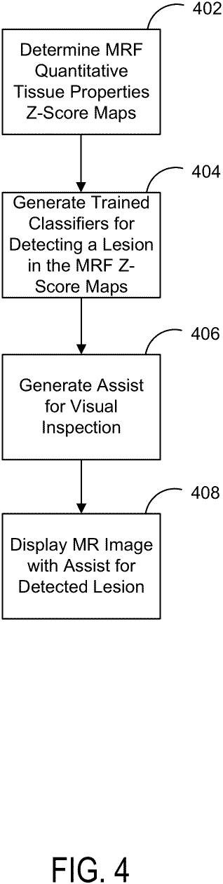

Referring to FIG. 4, non-limiting example steps are shown for automated lesion detection based upon generated z-score maps. MRF quantitative tissue properties z-score maps are determined at step 402. Z-scores may be generated following the description above, such as for FIG. 2, or FIG. 3. Quantitative tissue properties include T₁, T₂, M0, GM, WM, and the like. Trained classifiers may be generated at step 404. Training of a machine learning or deep learning classifier, or the like can be performed based on labeled patient data and control data. An assist for visual inspection may be generated at step 406. The assist may include a tagged or labeled portion of an MR image that highlights a lesion detected by the trained classifier for a user. The assisted or labeled MR image depicting the detected lesion identified by the trained classifier may be displayed at step 408 for a user. In some configurations, the image displayed for a user may include an MR image of the subject with the detected lesion overlaid or otherwise highlighted for a user’s attention.

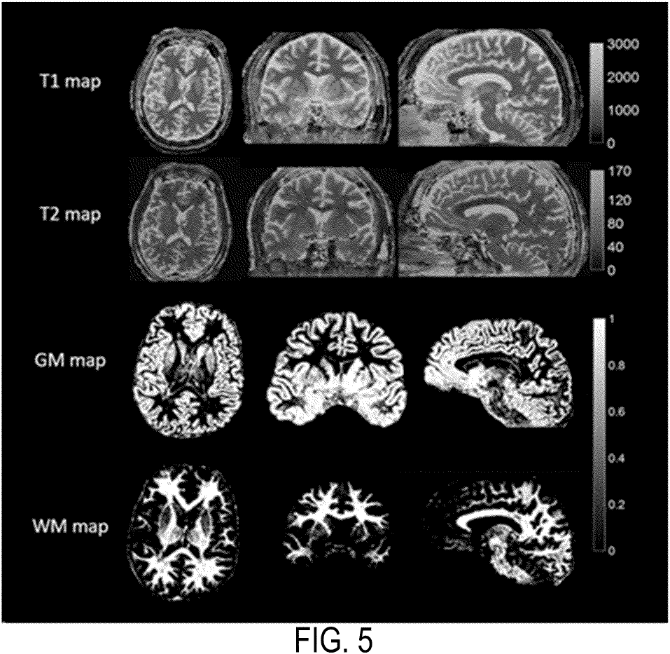

Referring to FIG. 5, non-limiting example MRF maps are shown for T1, T2, GM and WM. Quantitative values from these maps are used as input to generate the z-score maps to identify areas with the greatest differences when comparing to normal controls, in order to identify lesions.

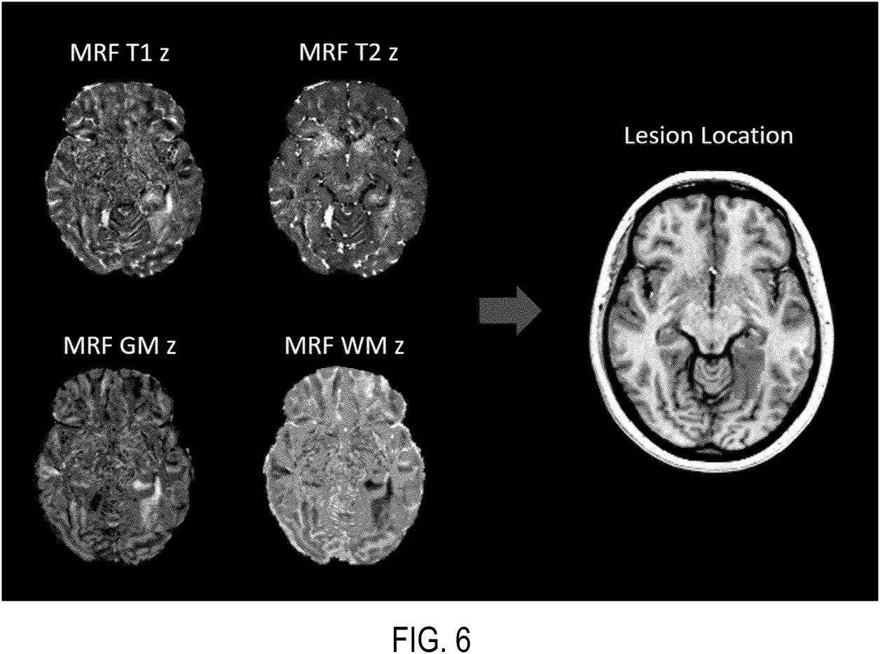

Referring to FIG. 6, non-limiting example original and processed images are shown from an epilepsy patient with focal cortical dysplasia (FCD), depicting an automatically identified lesion that may be localized by the conspicuous appearance in the processed images. The images were processed according to the above methods.

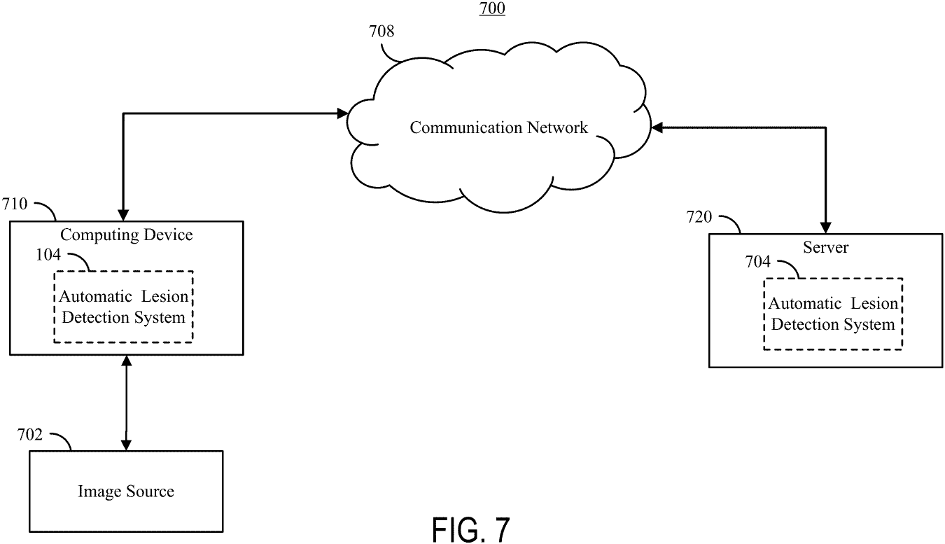

FIG. 7 shows an example 700 of a system for automatically detection lesions using image data in accordance with some embodiments of the disclosed subject matter. As shown in FIG. 7, a computing device 710 can receive multiple types of image data from an image source 702. In some configurations, computing device 710 can execute at least a portion of an automatic lesion detection system 704 to automatically determine whether epilepsy lesions are present in images of a subject.

Additionally or alternatively, in some embodiments, computing device 710 can communicate information about image data received from image source 702 to a server 720 over a communication network 708, which can execute at least a portion of automatic lesion detection system 704 to automatically determine whether epilepsy lesions are present in images of a subject. In such embodiments, server 720 can return information to computing device 710 (and/or any other suitable computing device) indicative of an output of automatic lesion detection system 704 to determine whether epilepsy lesions are present or absent.

In some embodiments, computing device 710 and/or server 720 can be any suitable computing device or combination of devices, such as a desktop computer, a laptop computer, a smartphone, a tablet computer, a wearable computer, a server computer, a virtual machine being executed by a physical computing device, etc. In some configurations, automatic lesion detection system 704 can extract features from labeled (e.g., labeled as including a lesion, condition, or disease, or normal) image data using a convolutional neural network (CNN) trained as a general image classifier, and can perform a correlation analysis to calculate correlations between the features corresponding to the image data and a database. In some embodiments, the labeled data can be used to train a classification model, such as a support vector machine (SVM), to classify features as indicative of a disease or a condition, or as indicative of normal. In some embodiments, automatic lesion detection system 704 can provide features for unlabeled image data to the trained classification model and can present a report or map based on the output of the classification model (e.g., based on which class the SVM identifies the features with).

In some embodiments, image source 702 can be any suitable source of image data, such as an MRI, etc. In some embodiments, image source 702 can be local to computing device 710. For example, image source 702 can be incorporated with computing device 710 (e.g., computing device 710 can be configured as part of a device for capturing and/or storing images). As another example, image source 702 can be connected to computing device 710 by a cable, a direct wireless link, etc. Additionally or alternatively, in some embodiments, image source 702 can be located locally and/or remotely from computing device 710, and can communicate image data to computing device 710 (and/or server 720) via a communication network (e.g., communication network 708).

In some embodiments, communication network 708 can be any suitable communication network or combination of communication networks. For example, communication network 708 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 708 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, etc.

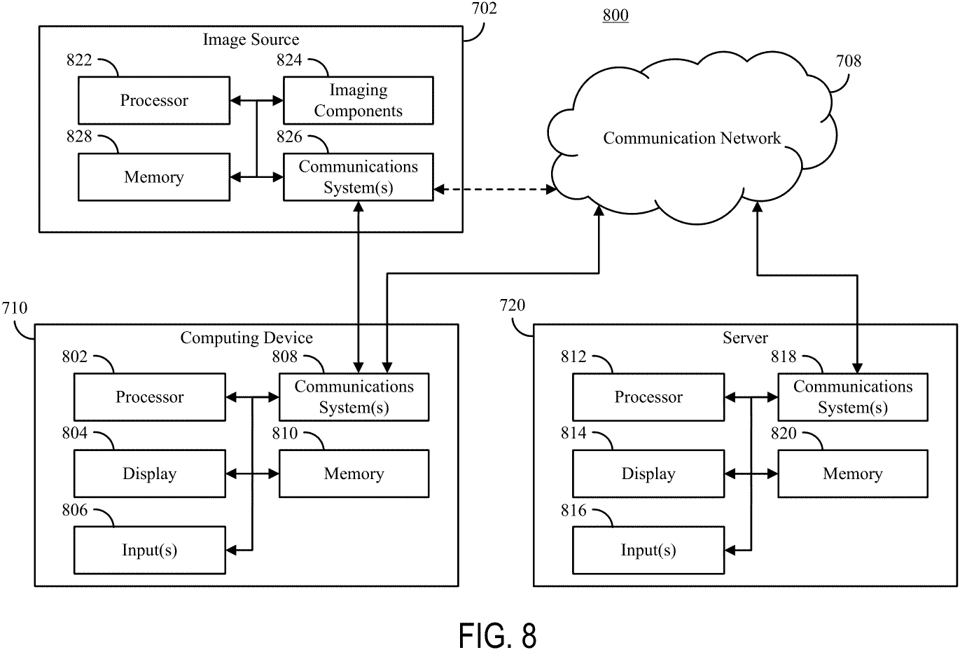

FIG. 8 shows an example 800 of hardware that can be used to implement image source 702, computing device 710, and/or server 720 in accordance with some embodiments of the disclosed subject matter. As shown in FIG. 8, in some embodiments, computing device 710 can include a processor 802, a display 804, one or more inputs 806, one or more communication systems 808, and/or memory 810. In some embodiments, processor 802 can be any suitable hardware processor or combination of processors, such as a central processing unit (CPU), a graphics processing unit (GPU), etc. In some embodiments, display 804 can include any suitable display devices, such as a computer monitor, a touchscreen, a television, etc. In some embodiments, inputs 806 can include any suitable input devices and/or sensors that can be used to receive user input, such as a keyboard, a mouse, a touchscreen, a microphone, etc.

In some embodiments, communications systems 808 can include any suitable hardware, firmware, and/or software for communicating information over communication network 708 and/or any other suitable communication networks. For example, communications systems 808 can include one or more transceivers, one or more communication chips and/or chip sets, etc. In a more particular example, communications systems 808 can include hardware, firmware and/or software that can be used to establish a Wi-Fi connection, a Bluetooth connection, a cellular connection, an Ethernet connection, etc.

In some embodiments, memory 810 can include any suitable storage device or devices that can be used to store instructions, values, etc., that can be used, for example, by processor 802 to present content using display 804, to communicate with server 720 via communications system(s) 808, etc. Memory 810 can include any suitable volatile memory, non-volatile memory, storage, or any suitable combination thereof. For example, memory 810 can include RAM, ROM, EEPROM, one or more flash drives, one or more hard disks, one or more solid state drives, one or more optical drives, etc. In some embodiments, memory 810 can have encoded thereon a computer program for controlling operation of computing device 710. In such embodiments, processor 802 can execute at least a portion of the computer program to present content (e.g., MRI images, user interfaces, graphics, tables, etc.), receive content from server 720, transmit information to server 720, etc.

In some embodiments, server 720 can include a processor 812, a display 814, one or more inputs 816, one or more communications systems 818, and/or memory 820. In some embodiments, processor 812 can be any suitable hardware processor or combination of processors, such as a CPU, a GPU, etc. In some embodiments, display 814 can include any suitable display devices, such as a computer monitor, a touchscreen, a television, etc. In some embodiments, inputs 816 can include any suitable input devices and/or sensors that can be used to receive user input, such as a keyboard, a mouse, a touchscreen, a microphone, etc.

In some embodiments, communications systems 818 can include any suitable hardware, firmware, and/or software for communicating information over communication network 708 and/or any other suitable communication networks. For example, communications systems 818 can include one or more transceivers, one or more communication chips and/or chip sets, etc. In a more particular example, communications systems 818 can include hardware, firmware and/or software that can be used to establish a Wi-Fi connection, a Bluetooth connection, a cellular connection, an Ethernet connection, etc.

In some embodiments, memory 820 can include any suitable storage device or devices that can be used to store instructions, values, etc., that can be used, for example, by processor 812 to present content using display 814, to communicate with one or more computing devices 710, etc. Memory 820 can include any suitable volatile memory, non-volatile memory, storage, or any suitable combination thereof. For example, memory 820 can include RAM, ROM, EEPROM, one or more flash drives, one or more hard disks, one or more solid state drives, one or more optical drives, etc. In some embodiments, memory 820 can have encoded thereon a server program for controlling operation of server 720. In such embodiments, processor 812 can execute at least a portion of the server program to transmit information and/or content (e.g., MRI data, results of automatic diagnosis, a user interface, etc.) to one or more computing devices 710, receive information and/or content from one or more computing devices 710, receive instructions from one or more devices (e.g., a personal computer, a laptop computer, a tablet computer, a smartphone, etc.), etc.

In some embodiments, image source 702 can include a processor 822, imaging components 824, one or more communications systems 826, and/or memory 828. In some embodiments, processor 822 can be any suitable hardware processor or combination of processors, such as a CPU, a GPU, etc. In some embodiments, imaging components 824 can be any suitable components to generate image data corresponding to one or more imaging modes (e.g., T1 imaging, T2 imaging, fMRI, etc.). An example of an imaging machine that can be used to implement image source 702 can include a conventional MRI scanner (e.g., a 1.5 T scanner, a 3 T scanner), a high field MRI scanner (e.g., a 7 T scanner), an open bore MRI scanner, a CT system, an ultrasound scanner and the like.

Note that, although not shown, image source 702 can include any suitable inputs and/or outputs. For example, image source 702 can include input devices and/or sensors that can be used to receive user input, such as a keyboard, a mouse, a touchscreen, a microphone, a trackpad, a trackball, hardware buttons, software buttons, etc. As another example, image source 702 can include any suitable display devices, such as a computer monitor, a touchscreen, a television, etc., one or more speakers, etc.

In some embodiments, communications systems 826 can include any suitable hardware, firmware, and/or software for communicating information to computing device 710 (and, in some embodiments, over communication network 708 and/or any other suitable communication networks). For example, communications systems 826 can include one or more transceivers, one or more communication chips and/or chip sets, etc. In a more particular example, communications systems 826 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, etc.

In some embodiments, memory 828 can include any suitable storage device or devices that can be used to store instructions, values, image data, etc., that can be used, for example, by processor 822 to: control imaging components 824, and/or receive image data from imaging components 824; generate images; present content (e.g., MRI images, a user interface, etc.) using a display; communicate with one or more computing devices 710; etc. Memory 828 can include any suitable volatile memory, non-volatile memory, storage, or any suitable combination thereof. For example, memory 828 can include RAM, ROM, EEPROM, one or more flash drives, one or more hard disks, one or more solid state drives, one or more optical drives, etc. In some embodiments, memory 828 can have encoded thereon a program for controlling operation of image source 702. In such embodiments, processor 822 can execute at least a portion of the program to generate images, transmit information and/or content (e.g., MRI image data) to one or more computing devices 710, receive information and/or content from one or more computing devices 710, receive instructions from one or more devices (e.g., a personal computer, a laptop computer, a tablet computer, a smartphone, etc.), or the like.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A method for automatically detecting a lesion in a brain of a subject, comprising:
accessing in magnetic resonance fingerprinting (MRF) data acquired from the subject;
registering the MRF data of the subject to a template space;
generating a normal template from normal images without a lesion in the template space;
generating a z-score map using the registered MRF data of the subject and the generated normal template;
subjecting the generated z-score map to a trained classifier that is trained to detect the lesion in the generated z-score map; and
displaying an image of the subject or a report with an indication of a location of the detected lesion.

2. The method of claim 1, further comprising generating white matter (WM), gray matter (GM), cerebrospinal fluid (CSF), T1, T2, or M0 maps from MRF data prior to generating the z-score map.

3. The method of claim 2, further comprising determining a mean and standard deviation for the normal template.

4. The method of claim 3, wherein generating the z-score map includes determining a constituent z-score map for each WM, GM and CSF map using the determined mean and standard deviation of the normal template.

5. The method of claim 4, wherein generating the z-score map includes using an expression of the form:
$${Z}_{s c o r e} = \frac{M R {F}_{N} - M R {F}_{P}}{M R {F}_{S D}}$$
where Z_{score} represents a pixel value in the z-score map, MRF_{N} represents the mean of a normal MRF value determined from the normal template, MRF_{P} represents a MRF value in the MRF data of the subject, and MRF_{SD} represents the standard deviation of the normal template MRF values.

6. The method of claim 1, further comprising removing skull tissues in the MRF data by skull stripping of the MRF data.

7. The method of claim 1, further comprising smoothing the z-score maps using Gaussian smoothing to reduce noise.

8. The method of claim 1, further comprising reducing false positives in the z-score maps using a mask of the MRF data.

9. The method of claim 1, wherein the lesion is an epilepsy lesion.

10. The method of claim 1, wherein the trained classifier has been trained on labeled subject data and control data.

11. A system for automatically detecting a lesion in magnetic resonance fingerprinting (MRF) data of a subject, comprising:
a computer system configured to:
i) access MRF data of a subject containing the lesion;
ii) register the MRF data of the subject to a template space;
iii) generate a normal template from normal images without a lesion in the template space;
iv) generate a z-score map using the registered MRF data of the subject and the generated normal template;
v) subject the generated z-score map to a trained classifier trained to detect the lesion in the generated z-score map; and
vi) display an image of the subject with the detected lesion.

12. The system of claim 11, wherein the computer system is further configured to generate white matter (WM), gray matter (GM), cerebrospinal fluid (CSF), T1, T2, or M0 maps from the MRF data, prior to generating the z-score map.

13. The system of claim 12, wherein the computer system is further configured to determine a mean and standard deviation for the normal template.

14. The system of claim 13, wherein the computer system is further configured to generate the z-score map by determining a constituent z-score map for each WM, GM and CSF map using the determined mean and standard deviation of the normal template.

15. The system of claim 14, wherein the computer system is further configured to generate the z-score map using an expression of the form:
$${Z}_{s c o r e} = \frac{M R {F}_{N} - M R {F}_{P}}{M R {F}_{S D}}$$
where Z_{score} represents a pixel value in the z-score map, MRF_{N} represents the mean of a normal MRF value determined from the normal template, MRF_{P} represents a MRF value in the MRF data of the subject, and MRF_{SD} represents the standard deviation of the normal template MRF values.

16. The system of claim 11, wherein the computer system is further configured to remove skull tissue in the MRF data by skull stripping of the MRF data.

17. The system of claim 11, wherein the computer system is further configured to smooth the z-score maps using Gaussian smoothing to reduce noise.

18. The system of claim 11, wherein the computer system is further configured to reduce false positives in the z-score maps using a mask of the MRF data.

19. The system of claim 11, wherein the lesion is an epilepsy lesion.

20. The system of claim 11, wherein the trained classifier has been trained on labeled subject data and control data.
